SEQUENCE LISTING (22 sequences)

SEQ ID NO: 1 - DNA, 705 bp, Artificial Sequence
  Human enterokinase
atcgtcggcggaagcaatgcgaaggaaggcgcctggccatgggtggtgggcctctactacggaggacgcctcctctgcggcgcctctctcgtgtccagcgactggctcgtgtccgccgctcactgcgtgtacggccgcaacctcgagccatccaagtggacagccatcctcggcctccacatgaagtccaacctcacctctccacagaccgtgccacgcctgatcgacgagatcgtgatcaacccacactacaaccgccgccgcaaggacaacgacatcgccatgatgcacctcgagttcaaggtgaactacaccgactacatccagccaatctgcctcccagaggagaaccaggtgttcccaccaggccgcaactgctccatcgctggctggggcaccgtggtgtaccaaggcaccaccgccaacatcctccaggaggccgacgtgccactcctctccaacgagcgctgccagcaacagatgccagaatacaacatcaccgaaaacatgatctgcgctggctacgaagaaggaggcatcgactcctgccagggcgactctggcggaccactcatgtgccaggagaacaaccgctggttcctcgctggcgtgacctccttcggctacaagtgcgctctcccaaaccgcccaggcgtgtacgccagggtgtctcgcttcaccgagtggattcagagcttcctgcat

SEQ ID NO: 2 - DNA, 861 bp, Artificial Sequence
  3D(RAmy3D)-Human enterokinase
ttggatccatcagtagtggttagcagcaactcactatcgaacacggtttcagcttacacagatatgaagaacaccagcagcttgtgtttgctgctcctcgtggtgctctgcagcttgacctgcaactcgggccaagcccaggtcctcatcgtcggcggaagcaatgcgaaggaaggcgcctggccatgggtggtgggcctctactacggaggacgcctcctctgcggcgcctctctcgtgtccagcgactggctcgtgtccgccgctcactgcgtgtacggccgcaacctcgagccatccaagtggacagccatcctcggcctccacatgaagtccaacctcacctctccacagaccgtgccacgcctgatcgacgagatcgtgatcaacccacactacaaccgccgccgcaaggacaacgacatcgccatgatgcacctcgagttcaaggtgaactacaccgactacatccagccaatctgcctcccagaggagaaccaggtgttcccaccaggccgcaactgctccatcgctggctggggcaccgtggtgtaccaaggcaccaccgccaacatcctccaggaggccgacgtgccactcctctccaacgagcgctgccagcaacagatgccagaatacaacatcaccgaaaacatgatctgcgctggctacgaagaaggaggcatcgactcctgccagggcgactctggcggaccactcatgtgccaggagaacaaccgctggttcctcgctggcgtgacctccttcggctacaagtgcgctctcccaaaccgcccaggcgtgtacgccagggtgtctcgcttcaccgagtggattcagagcttcctgcattgaggtacc

SEQ ID NO: 3 - DNA, 879 bp, Artificial Sequence
  3D(RAmy3D)-Human enterokinase-6His
ttggatccatcagtagtggttagcagcaactcactatcgaacacggtttcagcttacacagatatgaagaacaccagcagcttgtgtttgctgctcctcgtggtgctctgcagcttgacctgcaactcgggccaagcccaggtcctcatcgtcggcggaagcaatgcgaaggaaggcgcctggccatgggtggtgggcctctactacggaggacgcctcctctgcggcgcctctctcgtgtccagcgactggctcgtgtccgccgctcactgcgtgtacggccgcaacctcgagccatccaagtggacagccatcctcggcctccacatgaagtccaacctcacctctccacagaccgtgccacgcctgatcgacgagatcgtgatcaacccacactacaaccgccgccgcaaggacaacgacatcgccatgatgcacctcgagttcaaggtgaactacaccgactacatccagccaatctgcctcccagaggagaaccaggtgttcccaccaggccgcaactgctccatcgctggctggggcaccgtggtgtaccaaggcaccaccgccaacatcctccaggaggccgacgtgccactcctctccaacgagcgctgccagcaacagatgccagaatacaacatcaccgaaaacatgatctgcgctggctacgaagaaggaggcatcgactcctgccagggcgactctggcggaccactcatgtgccaggagaacaaccgctggttcctcgctggcgtgacctccttcggctacaagtgcgctctcccaaaccgcccaggcgtgtacgccagggtgtctcgcttcaccgagtggattcagagcttcctgcatcatcaccatcaccatcactgaggtacc

SEQ ID NO: 4 - DNA, 80 bp, Artificial Sequence
  primer
atcgtcggcggaagcaatgcgaaggaaggcgcctggccatgggtggtgggcctctactacggaggacgcctcctctgcgg

SEQ ID NO: 5 - DNA, 80 bp, Artificial Sequence
  Primer
ttgcggccgtacacgcagtgagcggcggacacgagccagtcgctggacacgagagaggcgccgcagaggaggcgtcctcc

SEQ ID NO: 6 - DNA, 80 bp, Artificial Sequence
  Primer
gaggtgaggttggacttcatgtggaggccgaggatggctgtccacttggatggctcgaggttgcggccgtacacgcagtg

SEQ ID NO: 7 - DNA, 80 bp, Artificial Sequence
  Primer
cggcggttgtagtgtgggttgatcacgatctcgtcgatcaggcgtggcacggtctgtggagaggtgaggttggacttcat

SEQ ID NO: 8 - DNA, 80 bp, Artificial Sequence
  Primer
tagtcggtgtagttcaccttgaactcgaggtgcatcatggcgatgtcgttgtccttgcggcggcggttgtagtgtgggtt

SEQ ID NO: 9 - DNA, 80 bp, Artificial Sequence
  Primer
gagcagttgcggcctggtgggaacacctggttctcctctgggaggcagattggctggatgtagtcggtgtagttcacctt

SEQ ID NO: 10 - DNA, 80 bp, Artificial Sequence
  Primer
ccaccaggccgcaactgctccatcgctggctggggcaccgtggtgtaccaaggcaccaccgccaacatcctccaggaggc

SEQ ID NO: 11 - DNA, 80 bp, Artificial Sequence
  Primer
gtgatgttgtattctggcatctgttgctggcagcgctcgttggagaggagtggcacgtcggcctcctggaggatgttggc

SEQ ID NO: 12 - DNA, 80 bp, Artificial Sequence
  Primer
gagtcgccctggcaggagtcgatgcctccttcttcgtagccagcgcagatcatgttttcggtgatgttgtattctggcat

SEQ ID NO: 13 - DNA, 80 bp, Artificial Sequence
  Primer
aaggaggtcacgccagcgaggaaccagcggttgttctcctggcacatgagtggtccgccagagtcgccctggcaggagtc

SEQ ID NO: 14 - DNA, 80 bp, Artificial Sequence
  Primer
gtgaagcgagacaccctggcgtacacgcctgggcggtttgggagagcgcacttgtagccgaaggaggtcacgccagcgag

SEQ ID NO: 15 - DNA, 45 bp, Artificial Sequence
  Primer
atgcaggaagctctgaatccactcggtgaagcgagacaccctggc

SEQ ID NO: 16 - DNA, 30 bp, Artificial Sequence
  Primer
ttggatccatcagtagtggttagcagcaac

SEQ ID NO: 17 - DNA, 27 bp, Artificial Sequence
  Primer
gaggacctgggcttggcccgagttgca

SEQ ID NO: 18 - DNA, 34 bp, Artificial Sequence
  Primer
caagcccaggtcctcatcgtcggcggaagcaatg

SEQ ID NO: 19 - DNA, 28 bp, Artificial Sequence
  Primer
ggtacctcaatgcaggaagctctgaatc

SEQ ID NO: 20 - DNA, 42 bp, Artificial Sequence
  Primer
ggtacctcagtgatggtgatggtgatgatgcaggaagctctg

SEQ ID NO: 21 - protein, 6 aa, Artificial Sequence
  the amino acid sequence of trypsinogen recognized by enterokinase
VDDDDK

SEQ ID NO: 22 - protein, 5 aa, Artificial Sequence
  the amino acid sequence cleaved by the enterokinase
DDDDK

(19) United States
(12) Patent Application Publication
(10) Pub. No.: US 2015/0020238 A1
(43) Pub. Date: Jan. 15, 2015

(54) Plant producing human enterokinase light chain protein and uses thereof

(71) Applicant: Tae Ho Kwon, Jeollabuk-do (KR)

(72) Inventor: Tae Ho Kwon, Jeollabuk-do (KR)

(73) Assignee: NBM CO., LTD., Jeollabuk-do (KR)

(21) Appl. No.: 14/365,634

(22) PCT Filed: Jul. 19, 2012
(86) PCT No.: PCT/KR2012/005757

§ 371 Date: Jul. 29, 2014

(30) Foreign Application Priority Data
Apr. 6, 2012 (KR) 10-2012-0036227

(51) Int. Cl. C12N 9/50; C12N 15/82

(52) U.S. Cl. CPC C12N 9/50; C12N 15/8257; C12Y 304/21009
USPC 800/288; 536/23.2; 435/320.1; 435/419; 435/219; 800/298; 800/320.2

(57) ABSTRACT

The present invention provides a synthetic gene encoding the human enterokinase light chain protein, a recombinant vector comprising the synthetic gene encoding the protein, a plant cell transformed with the recombinant vector, a method for producing the human enterokinase light chain protein in a plant by using the recombinant vector, a method for producing a plant producing the human enterokinase light chain protein by transforming a plant cell with the recombinant vector, a plant producing the human enterokinase light chain protein which is produced by the method, and a seed thereof, and a composition for large-scale production of the human enterokinase light chain protein in a plant, in which the composition comprises the synthetic gene encoding the human enterokinase light chain protein.

### CROSS REFERENCE TO RELATED APPLICATIONS AND CLAIM OF PRIORITY

This patent application is a National Phase application under 35 U.S.C. §371 of International Application No. PCT/KR2012/005757, filed Jul. 19, 2012, which claims priority to Korean Patent Application No. 10-2012-0036227, filed Apr. 6, 2012, entire contents of which are incorporated herein by reference.

### TECHNICAL FIELD

The present invention relates to a plant producing the human enterokinase light chain protein and uses thereof. More specifically, it relates to a synthetic gene encoding the human enterokinase light chain protein, in which codon usage is optimized for expression in a plant, a recombinant vector comprising the synthetic gene encoding the protein, a plant cell transformed with the recombinant vector, a method for producing the human enterokinase light chain protein in a plant by using the recombinant vector, a method for producing a plant producing the human enterokinase light chain protein by transforming a plant cell with the recombinant vector, a plant producing the human enterokinase light chain protein which is produced by the method and a seed thereof, and a composition for large-scale production of human enterokinase light chain protein in a plant comprising the synthetic gene encoding the human enterokinase light chain protein.

### BACKGROUND ART

To produce cells for the application to cell therapy, which recently receives a great attention as a new technique for treating disorders, it is necessary to use a specific protease. However, due to its intrinsic characteristics, the protease cannot be produced as a recombinant protein by gene expression, and thus trypsin or the like which is derived from an animal is purified and used.

However, there is a possibility of having secondary human infection caused by incorporation of an animal virus when a protease is purified from animal organs, and thus the use of protease has been limited. Specifically, use of an enzyme isolated and purified from organs of a cow is currently prohibited due to the problems involving mad cow disease or the like. Further, an endopeptidase for cleavage at specific site, which is essentially required for studying proteins used in industry or research area, is also difficult to be produced in recombinant protein form. However, it is expected that a demand for various endopeptidases for cleavage at specific site will significantly increase in future in accordance with development of biological engineering.

The enterokinase specifically recognizes and cleaves the amino acid sequence of trypsinogen, i.e., sequence of Val-Asp-Asp-Asp-Asp-Lys (SEQ ID NO:21), for converting it into trypsin and it activates various pancreatic zymogens (Kunitz, J. Gen. Physiol., 1939, 22:429-446). The amino acid sequence Asp-Asp-Asp-Asp-Lys (SEQ ID NO:22) cleaved by the enterokinase is preserved in many vertebrates as a recognition site for enterokinase. Due to high specificity to the amino acid sequence described as Asp-Asp-Asp-Asp-Lys (SEQ ID NO:22), the enterokinase is widely used for site-specific cleavage of a fusion protein (LaVallie et al., J. Biol. Chem., 1993, 268:23311-17).

It is extremely difficult to produce the enterokinase, which is a medical protein, in recombinant protein form based on gene expression. Thus, it is an expensive enzyme (2,000,000 Won/500 IU). In addition to being used for medical use, it is also an essential enzyme used for removing Tag protein such as GST and 6× His that are generally used for producing a recombinant medical protein (Yuan and Hua, Protein Expression and Purification, 2002, 25:300304). Although the proteases like trypsin which have been known until now can be directly used as a medical or industrial protein material, as they are an enzyme necessarily required for protein processing for activating the functions of other proteins, high demand for them exists. However, as they can cause suppressed growth or death of host cells due to the intrinsic characteristics of the enzyme, their production has been often limited. To overcome such problems, it is tried to produce a recombinant protein by changing the host. However, it is currently impossible to produce the recombinant protein based on gene expression. At present, when the enterokinase gene is expressed in E. coil, it is expressed in inclusion body form, and there is a problem that the expressed protein is added at the amino terminal with an additional amino acid so that the activity is lowered (Collins-Racie et al., Bio/technology, 1995, 13:982-987). To solve such problems, the enterokinase is produced by animal cell culture. However, because the yield is extremely poor, production of recombinant protein by using microorganisms or by animal cell culture is very difficult to achieve. As such, some are isolated and purified from organs of a pig or a goat, and used.

Meanwhile, in Korean Patent Registration No. 0507980, “Recombinant enterokinase with modified amino terminal or carboxy terminal of enterokinase light chain” is disclosed. Further, in Korean Patent Application Publication No. 1999-0008525, “Novel enterokinase light chain (EKL) gene, nucleotide sequence thereof, E. coli expression vector and yeast expression vector for producing EKL, and method for producing EKL using them” is disclosed. However, the plant producing the human enterokinase light chain protein and uses thereof as disclosed in the present invention have never been described before.

### SUMMARY

The present invention is devised in view of the needs described above, and inventors of the present invention established a method for large-scale production of enterokinase light chain protein including incorporating a recombinant vector which comprises a synthetic gene encoding the human enterokinase light chain protein to a plant followed by overexpression and isolating and purifying the recombinant enterokinase light chain protein. According to the present invention, transformed plant cells that are incorporated with a recombinant gene are cultured in the same manner as culture of microorganisms by utilizing techniques of molecular biology, cellular engineering, biological processes, and isolation and purification among the techniques of biological engineering, and thus a technique for large-scale production of the enterokinase light chain protein, which is a kind of proteases and a non-producible protein, by using plant cells having a cell wall to protect the cells unlike microorganisms and animal cell culture is established, and the present invention is completed accordingly.

In order to solve the problems described above, the present invention provides a synthetic gene encoding the human enterokinase light chain protein.

The present invention further provides a recombinant vector comprising the synthetic gene encoding the human enterokinase light chain protein.

The present invention further provides a plant cell transformed with the recombinant vector.

The present invention further provides a method for producing the human enterokinase light chain protein in a plant by using the recombinant vector.

The present invention further provides a method for producing a plant producing the human enterokinase light chain protein by transforming a plant cell with the recombinant vector.

The present invention further provides a plant producing the human enterokinase light chain protein which is produced by the aforementioned method, and a seed thereof.

The present invention still further provides a composition for large-scale production of human enterokinase light chain protein in a plant comprising the synthetic gene encoding the human enterokinase light chain protein.

When the enterokinase gene is expressed in E. coli, there has been a problem that it is expressed in the inclusion body form and the activity is lowered due to addition of extra amino acids to the amino terminal Further, when the enterokinase is produced by animal cell culture, there has been a problem that the yield is low and, in case of the protein produced by animal cells, secondary human infection may occur due to incorporation of an animal virus. The plant cell culture system of the present invention is, however, a new production system which overcomes such problems. Unlike culture of microorganisms, the plant cell culture system does not involve production in inclusion body form. Instead, production is made completely in water-soluble form, thus showing high productivity. Further, although the protein produced by animal cells has a possibility of inducing secondary human infection as caused by animal viruses, a plant cell does not have any death of host cell caused by proteases produced since it has a cell wall. In addition, by having completely no possibility of inducing secondary human infection caused by plant viruses, the aforementioned system is believed to be an optimum system for large-scale production of enterokinase.

### BRIEF DESCRIPTION OF THE DRAWINGS

FIG. 1 illustrates the results of determining the final DNA fragment synthesized by using overlap PCR.

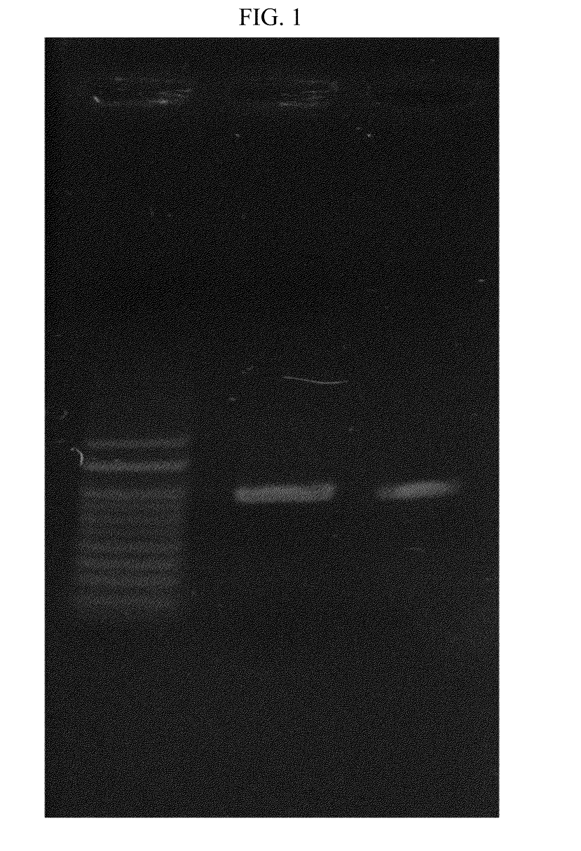

FIG. 2 illustrates a schematic diagram showing the plant expression vector pJKF39 and pJKF40 which include the gene encoding the human enterokinase light chain protein.

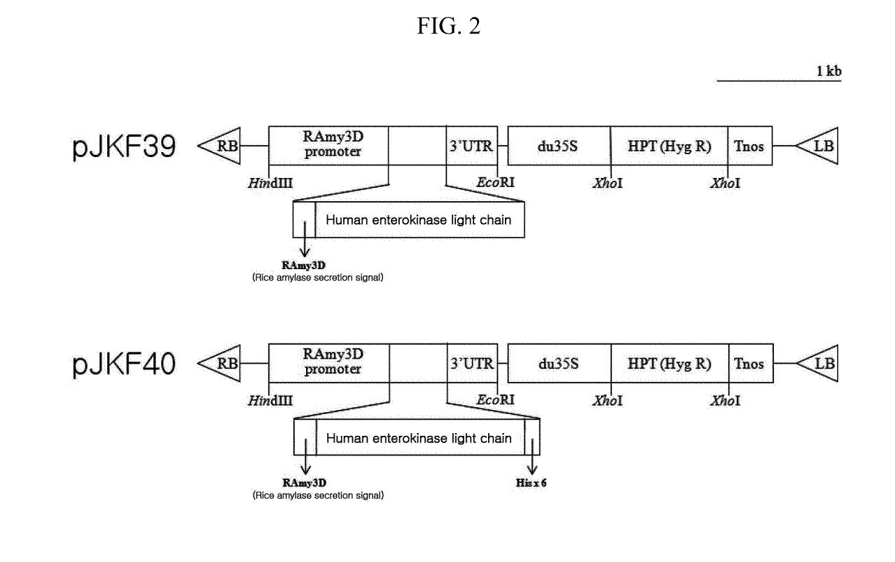

FIG. 3 illustrates the callus of transformed rice and the callus of rice under proliferation after transformation.

FIG. 4 illustrates the results of determining the presence or absence of transformed rice callus which has been transformed with pJKF39 or pJKF40, in which the determination is made by genomic DNA PCR.

FIGS. 5A to 5D illustrate the results of Western blot analysis performed for the plant cell line which has been transformed with pJKF39 for producing the human enterokinase light chain protein, in which the transformation has been carried out by using gene gun (FIG. 5A) or Agrobacterium (FIG. 5B), and the results of Western blot analysis performed for the plant cell line which has been transformed with pJKF40 for producing the human enterokinase light chain protein, in which the transformation has been carried out by using gene gun (FIG. 5C) or Agrobacterium (FIG. 5D).

FIGS. 6A and 6B illustrate the results of Northern blot analysis performed for the plant cell line which has been transformed with pJKF39 for producing the human enterokinase by using a gene gun (FIG. 6A), and the plant cell line which has been transformed with pJKF40 for producing the human enterokinase by using Agrobacterium (FIG. 6B).

FIGS. 7A and 7B illustrate the structure of EGFP-hTNF-α fusion protein comprising the enterokinase-recognizing sequence (FIG. 7A) and the results of determining the endopeptidase activity of the enzyme (FIG. 7B), which has been produced in a plant cell line producing the human enterokinase light chain protein, by using the EGFP-hTNF-α fusion protein.

FIGS. 8A and 8B illustrate suspended transformed cells with a volume of 50 ml (FIG. 8A) and culture of the suspended transformed cells with a volume of 300 ml (FIG. 8B).

FIGS. 9A to 9C illustrate the young plant regenerated from the transformed callus (FIG. 9A), the grown plant after regeneration (FIG. 9B), and the pJKF40 b4 plant (FIG. 9C) which has been incorporated with the gene encoding the enterokinase light chain protein.

FIG. 10 illustrates the results of confirming the cellular activity after cryopreservation of the cell line expressing the enterokinase light chain protein at high level.

FIG. 11 illustrates the establishment of the large-scale production system for the cell line expressing the enterokinase light chain protein at high level (left column) and the transformed cells being cultured in a bioreactor (right column).

FIG. 12 illustrates the UV peaks of the enterokinase light chain protein according to the purification which uses size exclusion chromatography and FPLC.

### DETAILED DESCRIPTION

In order to achieve the purpose of the invention, the present invention provides a synthetic gene encoding the human enterokinase light chain protein.

With regard to the synthetic gene encoding the human enterokinase light chain protein of the present invention, the codon usage of the human enterokinase gene was analyzed and obtained from GenBank, and the nucleotide sequences were designed and primers were synthesized after comparison with the codon usage in rice, and the enterokinase gene was synthesized based on overlap PCR.

The synthetic gene encoding the human enterokinase light chain protein of the present invention may consist of the nucleotide sequence of SEQ ID NO: 1, but it is not limited thereto. Further, variants of the nucleotide sequence are also within the scope of the present invention. Specifically, the above described gene may comprise a nucleotide sequence which has preferably at least 70%, more preferably at least 80%, still more preferably at least 90%, and most preferably at least 95% homology with the nucleotide sequence of SEQ ID NO: 1. The “sequence homology %” for a certain polynucleotide is identified by comparing a comparative region with two sequences that are optimally aligned. In this regard, a part of the polynucleotide in comparative region may comprise an addition or a deletion (i.e., a gap) compared to a reference sequence (without any addition or deletion) relative to the optimized alignment of the two sequences.

According to the gene of one embodiment of the present invention, the synthetic gene may additionally comprise a gene encoding the rice alpha amylase 3D (RAmy3D) secretion signal at the 5′-terminal The synthetic gene encoding the enterokinase light chain protein, which additionally comprises a gene encoding the rice alpha amylase 3D (RAmy3D) secretion signal at the 5′-terminal, may preferably consist of SEQ ID NO: 2, but it is not limited thereto.

According to the gene of one embodiment of the present invention, the synthetic gene may additionally comprise a gene encoding 6× His at the 3′-terminal

The synthetic gene encoding the enterokinase light chain protein, which additionally comprises the gene encoding 6× His at the 3′-terminal and a gene encoding the rice alpha amylase 3D (RAmy3D) secretion signal at the 5′-terminal, may preferably consist of SEQ ID NO: 3, but it is not limited thereto. Meanwhile, having additionally the gene encoding 6× His at the 3′-terminal is only to facilitate the isolation and purification of the human enterokinase light chain protein after expression in a host cell, and it has no effect on the activity of the protein.

The present invention further provides a recombinant vector comprising the synthetic gene encoding the human enterokinase light chain protein.

The term “recombinant” indicates a cell which replicates a heterogeneous nucleotide or expresses said nucleotide, or a peptide, a heterogeneous peptide, or a protein encoded by a heterogeneous nucleotide. Recombinant cell can express a gene or a gene fragment in the form of a sense or antisense, which are not found in natural state of cell. In addition, a recombinant cell can express a gene that is found in natural state, provided that said gene is modified and re-introduced into the cell by an artificial means.

The expression vector comprising the synthetic gene sequence encoding the enterokinase light chain protein and suitable transcription/translation regulating signals can be constructed by a method well known to a person skilled in the art. It includes an in vitro recombinant DNA technique, a technique for DNA synthesis, and an in vivo recombination technique. To induce mRNA synthesis, the DNA sequence may be effectively linked to a suitable promoter in an expression vector. Further, the expression vector may comprise, as a translation initiation region, a ribosome binding site and a transcription terminator.

Preferred example of the recombinant vector of the present invention is Ti-plasmid vector which can transfer a part of itself, i.e., so called T-region, to a plant cell when the vector is present in an appropriate host such as Agrobacterium tumefaciens. Other types of Ti-plasmid vector (see, EP 0 116 718 B1) are currently used for transferring a hybrid DNA sequence to protoplasts that can produce a new plant by appropriately inserting a plant cell or hybrid DNA to a genome of a plant. Especially preferred form of Ti-plasmid vector is a so-called binary vector which has been disclosed in EP 0 120 516 B1 and U.S. Pat. No. 4,940,838. Other vector that can be used for introducing the DNA of the present invention to a host plant can be selected from a double-stranded plant virus (e.g., CaMV), a single-stranded virus, and a viral vector which can be originated from Gemini virus, etc., for example a non-complete plant viral vector. Use of said vector can be advantageous especially when a host plant cannot be easily transformed.

Expression vector may comprise at least one selective marker. Said selective marker is a nucleotide sequence having a property of being selected by a common chemical method. Examples include all genes that are useful for distinguishing transformed cells from non-transformed cells. Specific examples thereof include a gene resistant to herbicide such as glyphosate and phosphinotricine, a gene resistant to antibiotics such as kanamycin, G418, bleomycin, hygromycin, and chloramphenicol, and aadA gene, but not limited thereto.

For the recombinant vector according to the present invention, the promoter can be any of rice amylase 3D (RAmy3D), CaMV 35S, actin, ubiquitin, pEMU, MAS, histone promoter, and C1p promoter, but not limited thereto. The term “promoter” means a DNA molecule to which RNA polymerase binds in order to initiate its transcription, and it corresponds to a DNA region upstream of a structural gene. The term “plant promoter” indicates a promoter which can initiate transcription in a plant cell.

For the recombinant vector of the present invention, any conventional terminator can be used. Examples include nopaline synthase (NOS), rice α-amylase RAmyl A terminator, phaseoline terminator, a terminator for octopine gene of Agrobacterium tumefaciens, and rnnB1/B2 terminator of E. coli, but are not limited thereto. Regarding the necessity of terminator, it is generally known that such region can increase reliability and an efficiency of transcription in plant cells. Therefore, the use of terminator is highly preferable in view of the contexts of the present invention.

The present invention further provides a plant cell transformed with the recombinant vector.

The host cell to be transformed with the vector of the present invention is preferably a plant cell, and more preferably rice.

The method to deliver the vector of the present invention to a host cell can be performed by using Agrobacterium-mediated transformation, gene bombardment, a microinjection method, calcium phosphate precipitation method, an electroporation method, a liposome-mediated transfection method, DEAE-dextran treatment method, and the like.

The present invention further provides a method for producing the human enterokinase light chain protein in a plant, comprising:

introducing the recombinant vector of the present invention to callus of a plant and selecting the callus which produces the human enterokinase light chain protein;

performing suspension culture of the selected callus; and

isolating and purifying the human enterokinase light chain protein from suspension culture liquid.

According to the method of one embodiment of the present invention, the plant is preferably a monocot plant such as rice, corn, wheat, barely, millet, rye, rye meal, sorghum, or oat, and more preferably rice, but it is not limited thereto.

For the isolation and purification of the human enterokinase light chain protein which has been produced by the plant as described in the present invention, various processes are applied according to a method which is easily used by a person skilled in the art as long as it remains within the technical scope of the present invention. The protein expressed from the recombinant molecule is obtained as a product which is purified from culture of the host cell for expression. Several steps of purification including washing, centrifugation, filtration, ultrafiltration, ammonium sulfate precipitation, use of silica beads, continuous centrifugation, rate zonal gradient centrifugation, and various chromatographic methods such as gel permeation, size exclusion, affinity, or ion exchange are generally employed. Preferably, the isolation and purification of the human enterokinase light chain protein can be performed by ammonium sulfate precipitation, hydrophobic interaction chromatography, and ion exchange chromatography in the order, but it is not limited thereto.

The present invention further provides a human enterokinase light chain protein produced by the method described above.

The present invention further provides a method for producing a plant producing the human enterokinase light chain protein comprising transforming a plant cell with the recombinant vector which comprises a synthetic gene encoding the human enterokinase light chain; and regenerating the plant from a transformed plant.

According to the production method of one embodiment of the present invention, the synthetic gene encoding the human enterokinase light chain protein preferably comprises the nucleotide sequence of SEQ ID NO: 1, a synthetic gene consisting of SEQ ID NO: 2 for encoding the enterokinase light chain protein, which additionally comprises a gene encoding the rice alpha amylase 3D (RAmy3D) secretion signal at the 5′-terminal, or a synthetic gene consisting of SEQ ID NO: 3 for encoding the enterokinase light chain protein, which additionally comprises the gene encoding 6× His at the 3 ‘-terminal and a gene encoding the rice alpha amylase 3D (RAmy3D) secretion signal at the 5’-terminal, but it is not limited thereto.

The method of the present invention includes a step of transforming a plant cell with the recombinant vector of the present invention and the transformation can be gene bombardment or Agrobacterium mediated transformation. The method of the present invention further includes a step of regenerating a transgenic plant from the transformed plant cell. As for the method for regenerating a transgenic plant from a transformed plant cell, any method known in the field can be used.

The present invention further provides a plant producing the human enterokinase light chain protein which is produced by the aforementioned method, and a seed thereof.

According to the plant of one embodiment of the present invention, the plant is preferably a monocot plant such as rice, corn, wheat, barley, millet, rye, rye meal, sorghum, or oat, and more preferably rice, but it is not limited thereto.

The present invention still further provides a composition for large-scale production of the human enterokinase light chain protein in a plant comprising a synthetic gene encoding the human enterokinase light chain protein.

According to the composition of one embodiment of the present invention, the synthetic gene encoding the human enterokinase light chain protein preferably comprises the nucleotide sequence of SEQ ID NO: 1, a synthetic gene consisting of SEQ ID NO: 2 for encoding the enterokinase light chain protein, which additionally comprises a gene encoding the rice alpha amylase 3D (RAmy3D) secretion signal at the 5′-terminal, or a synthetic gene consisting of SEQ ID NO: 3 for encoding the enterokinase light chain protein, which additionally comprises the gene encoding 6× His at the 3′-terminal and a gene encoding the rice alpha amylase 3D (RAmy3D) secretion signal at the 5′-terminal, but it is not limited thereto. The composition comprises, as an effective component, the synthetic gene encoding the human enterokinase light chain protein, and the human enterokinase light chain protein can be produced in a large amount according to transformation of a plant with said gene.

Hereinbelow, the present invention is explained in view of the examples. However, the following examples are given only to illustrate the present invention and by no means the scope of the present invention is limited to them.

### EXPERIMENTAL PROCEDURES

### 1. Gene Synthesis

By using the gene analysis program DNASIS, codon usage of the gene encoding the human enterokinase light chain protein was analyzed. After the obtainment from GenBank, the nucleotide sequence was designed and the primers were synthesized based on the comparison of codon usage in rice by using codon usage data base (http://www.kazusa.or.ip/codon/index.html) of Kazusa DNA Research Institute. In addition, the gene encoding the enterokinase light chain protein of the present invention was synthesized by overlap PCR.

### 2. Construction of Plant Expression Vector

By introducing the gene encoding the human enterokinase light chain protein, which has been synthesized by overlap PCR, to the plant expression vector pMYN75, a plant expression vector used for production of the human enterokinase light chain protein was constructed.

### 3. Plant Transformation

For transformation of rice (Oryza sativa L.), the callus derived from blastodisc which has been formed after in vitro germination of the seeds for a week or so was used. Two methods were employed for the transformation, i.e., transformation of callus using gene gun (particle-bombardment) and transformation using Agrobacterium, which is soil bacteria. When the gene gun method is used, 7 to 10 day-old calluses were used. On the other hand, for the Agrobacterium transformation, calluses cultured for 3 to 4 weeks were inoculated to Agrobacterium solution for 10 minutes. Then, the Agrobacterium on a surface of the explant was blotted on a sterilized filter paper and applied to a medium followed by ex vivo culture for three days in a dark place at 28° C. After the ex vivo culture, they were washed several times with DW and DW added with cefotaxime. After removing the moisture, they were planted on N6 selection medium added with hygromycin. The culture environment was controlled such that the temperature is 25° C. and the light/dark cycle is 8/16 hours. The grown callus was used as a material for confirming the incorporation of the gene encoding the enterokinase light chain protein and it was sub-cultured once per month.

### 4. Establishment of Cell Line Expressing Enterokinase Light Chain Protein at High Level

### (1) PCR and Western Blot Analysis

In order to confirm stable incorporation of the gene encoding the human enterokinase light chain protein to the callus selected from N6 selection medium, the genomic DNA was isolated from the transformed callus, and PCR analysis was performed by using a primer which is specific to the gene encoding the human enterokinase light chain protein. After homogenizing a small amount of the callus, the genomic DNA was extracted via a series of process and PCR was performed with 200 ng of the DNA. For the PCR, the forward primer and the reverse primer for the human enterokinase light chain protein were used and a process including pre-denaturation at 94° C. for 5 minutes, denaturation for 30 seconds at 94° C., primer-annealing for 30 seconds at 50° C., and elongation of the primer for 1 minute at 72° C. was repeated for 30 times. At the end of the process, the reaction was performed for 3 minutes at 72° C. The amplified product was subjected to electrophoresis using 1% agarose gel. According to staining with EtBr and determining the band under a UV lamp, suspension culture was performed with the identified callus. In order to confirm the expression of the human enterokinase light chain protein, the suspension culture cells were cultured for 5 days in N6 medium not added with sucrose. Then, by using the culture liquid, SDS-PAGE and Western blot analysis were performed.

### (2) RNA Extraction and Northern Blot Analysis

In order to see whether or not the expression of the gene encoding the human enterokinase light chain protein occurs without any problem in the callus, which has been confirmed with the incorporation of the gene encoding the human enterokinase light chain protein by genomic PCR analysis, the Northern blot analysis was performed. After culturing 5 days the cells obtained by the suspension culture from which the incorporation of the gene encoding the human enterokinase light chain protein has been confirmed in N6 medium not added with sucrose, callus was harvested and RNA was extracted with Tri-reagent. 30 μg was then quantified and subjected to electrophoresis using formaldehyde agarose gel. By capillary transfer, it was transferred onto Hybond N⁺-membrane and hybridized with a probe, i.e., the α³²P-dCTP labeled DNA encoding the human enterokinase light chain protein.

### 5. Establishment of Suspension Cells Expressing Enterokinase Light Chain Protein at High Level

By using the callus which has been confirmed with the expression of the gene encoding the human enterokinase light chain protein according to Western blot analysis and Northern blot analysis, suspension culture was performed. Specifically, after planting the callus in N6 selection medium not added with agar and allowing it to grow for 2 to 3 weeks, only the fine cells were collected by using a 1 mm×1 mm stainless mesh. With an interval of one week, sub-culture was performed in 50 ml volume. Further, the culture was performed by using a shaking incubator adjusted to have culture temperature of 25° C. and 90 rpm.

6. Plant Regeneration from Cell Line Expressing Enterokinase Light Chain Protein at High Level

The callus producing the enterokinase light chain protein at high level was planted on Ms-Re medium (MS medium prepared to have 5% sucrose, 2% sorbitol, 2 ppm kinetin, 1 ppm NAA, and 1.6% agar, and adjusted to pH 5.8) to induce new shoots and roots. The regenerated plant was transplanted in a pot after undergoing an acclimation process. While maintaining the growth condition at optimum conditions, growth to a healthy plant and seed formation were induced.

### 7. Establishment of Cell Bank of Cell Line Expressing Enterokinase Light Chain Protein at High Level

According to the cryopreservation method for suspension cells which has been previously created by the inventors of the present invention, the cell bank of cell line expressing enterokinase light chain protein at high level was established.

### 8. Large-Scale Culture of Cell Line Expressing Enterokinase Light Chain Protein at High Level

### (1) Large-Scale Culture of Cell Line Expressing Enterokinase Light Chain Protein at High Level

After increasing the volume of the suspension cells which have been established in 50 ml volume, large-scale culture was performed by using a 10 liter air bubble type bioreactor. Specifically, 100 ml of the cells were added to a specially manufactured 10 liter bioreactor, and 5 liter of N6 medium was added thereto. Seven days later, the medium was exchanged with the same but fresh medium not added with sucrose. The cells were harvested five days later. After the harvest, the culture liquid and the culture cells were collected and used as a material for analyzing the presence or absence of the expression of the enterokinase light chain protein.

### (2) Isolation and Purification of Enterokinase Light Chain Protein

For the isolation and purification of the enterokinase light chain protein which has been cultured and secreted with use of a bioreactor, three-step purification including ammonium sulfate precipitation, hydrophobic interaction chromatography using phenyl Sepharose 6FF resin, and ion exchange chromatography using mono S and mono Q was performed. As a result, the enterokinase light chain protein which has been produced by suspension cell culture of the transformed rice was isolated and purified.

### EXAMPLE 1

### Gene Synthesis

To the primer shEK-F1 (SEQ ID NO: 4) of Table 1, shEK-R1 (SEQ ID NO: 5), shEK-R2 (SEQ ID NO: 6), shEK-R3 (SEQ ID NO: 7), shEK-R4 (SEQ ID NO: 8), and shEK-R5 (SEQ ID NO: 9) were linked in order by a polymerization reaction. Also, to the primer shEK-F2 (SEQ ID NO: 10), shEK-R6 (SEQ ID NO: 11), shEK-R7 (SEQ ID NO: 12), shEK-R8 (SEQ ID NO: 13), shEK-R9 (SEQ ID NO: 14), and shEK-R10 (SEQ ID NO: 15) were linked in order by a polymerization reaction. Then, by a polymerization reaction of each fragment, the DNA fragment of 705 bp, which corresponds to a synthetic gene encoding the human enterokinase light chain protein with optimized rice codon, was obtained (SEQ ID NO: 1). It was then cloned into pGEM-T Easy vector and used for preparing pJKFEK which contains the synthesized gene encoding the human enterokinase light chain protein. As a result of determining the nucleotide sequence, correct synthesis was confirmed. For having extra-cellular secretion of the human enterokinase light chain protein which has been expressed in rice cells, pMYN44 (Shin et al., J. Biotechnology and Bioengineering, 2003, 82:-778783) comprising the signal sequence for secretion of rice alpha amylase 3D (RAmy3D) protein was amplified by using the primers 3Dsp-F (SEQ ID NO: 16) and 3Dsp-R (SEQ ID NO: 17) that are listed in Table 1. As a result, the DNA fragment of the signal sequence for secretion of rice alpha amylase 3D (RAmy3D) protein was obtained. Thereafter, for fusion of the signal sequence for secretion of rice alpha amylase 3D (RAmy3D) protein to the human enterokinase light chain protein, the DNA fragment of 729 bp, which has been obtained by amplifying the gene fragment of 705 bp with the primers 3Dsp-EK-F (SEQ ID NO: 18) and EK(KpnI)-R (SEQ ID NO: 19) and the 147 bp DNA fragment of signal sequence for secretion of rice alpha amylase 3D (RAmy3D) protein with SEQ ID NO: 19 were admixed with each other followed by amplification using the primers 3Dsp-F (SEQ ID NO: 16) and EK(KpnI)-R (SEQ ID NO: 19) to add, to the 5′ side of the gene encoding the human enterokinase light chain protein, the nucleotide sequence corresponding to the secretion signal of the rice alpha amylase 3D (RAmy3D) protein. It was then cloned into pGEM-T Easy vector and used for preparing pJKF3DEK. As a result of determining the nucleotide sequence, it was found that the gene of SEQ ID NO: 2 encoding the human enterokinase light chain protein was correctly synthesized with the 861 bp nucleotide sequence while including the secretion signal of the rice alpha amylase 3D (RAmy3D) protein. pJKF3DEK was amplified with the primers 3Dsp-F (SEQ ID NO: 16) and EK(His6)-R (SEQ ID NO: 20) to obtain the fragment of 879 bp. It was then cloned into pGEM-T Easy vector and used for preparing pJKF3DEKHis. As a result of determining the nucleotide sequence, it was found that the gene of SEQ ID NO: 3 encoding the human enterokinase light chain protein was correctly synthesized, in which six histidines are included at the carboxy terminal of the human enterokinase light chain protein containing the secretion signal of the rice alpha amylase 3D (RAmy3D) protein.

### [Table 1]

### EXAMPLE 2

### Construction of Plant Expression Vector

pJKF3DEK was digested with the restriction enzymes BamHI and KpnI. After isolating by agarose gel electrophoresis the gene fragment encoding the human enterokinase light chain protein, which includes the secretion signal for rice alpha amylase 3D (RAmy3D) of 857 bp, it was introduced to the plant expression vector pMYN75 after digestion with the restriction enzymes BamHI and KpnI. As a result, the plant expression vector pJKF39 for producing the human enterokinase light chain (HEK_{L}) protein was produced. Further, pJKF3DEKHis was digested with the restriction enzymes BamHI and KpnI. After isolating by agarose gel electrophoresis the gene fragment encoding the human enterokinase light chain protein, which includes the secretion signal for rice alpha amylase 3D (RAmy3D) of 877 bp, it was introduced to the plant expression vector pMYN75 obtained by digestion with the restriction enzymes BamHI and KpnI. As a result, the plant expression vector pJKF40 for producing the human enterokinase light chain (HEK_{L}) protein having six His tags at the C-terminal was produced. The pMYN75 vector used in the present examples is a rice expression vector comprising the rice alpha amylase 3D (RAmy3D) promoter, which is very strongly expressed when the suspension cells are in a carbohydrate-deficient state (Shin et al., J. Biotechnology and Bioengineering, 2003, 82:778-783).

### EXAMPLE 3

### Transformation of Plant

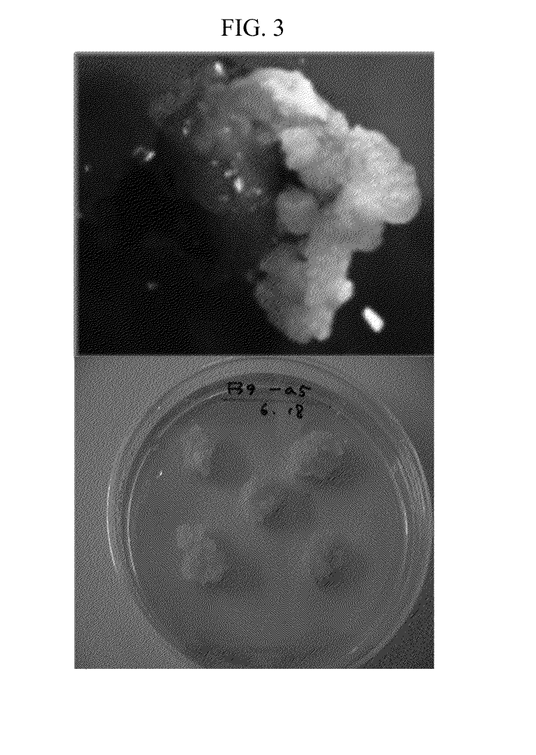

pJKF39 and pJKF40 were introduced to rice callus by using PDS-1000/He Biolistic Particle Delivery system of Bio-Rad Laboratories, Inc. and Agrobacterium transformation method. After the inoculating for 10 minutes the Agrobacterium tumefacience comprising the recombinant vector which has been produced to introduce the gene encoding enterokinase light chain protein to the rice callus, in vitro culture was performed for three days. After the in vitro culture, sub-culture was performed on a selection medium added with hygromycin. As a result, it was possible to confirm the normal growth of the callus. In addition, by using 67 lines in total including the plant transformed by gene gun method, the transformed pJKF39 line obtained by using (Agrobacterium) (pJKF39 a1˜9), the transformed pJKF39 line obtained by gene gun method (pJKF39 b1˜30), the transformed pJKF40 line obtained by using Agrobacterium (pJKF40 a1˜12), and the transformed pJKF40 line obtained by gene gun method (pJKF40 b1˜16) were obtained. FIG. 3 is an image showing the growing callus. According to sub-culture once per month on a selection medium, the proliferation was continued and a healthy callus with pale yellow color was obtained (FIG. 3).

### EXAMPLE 4

### Establishment of Cell Line Expressing Enterokinase Light Chain Protein at High Level

### (1) PCR and Western Blot Analysis

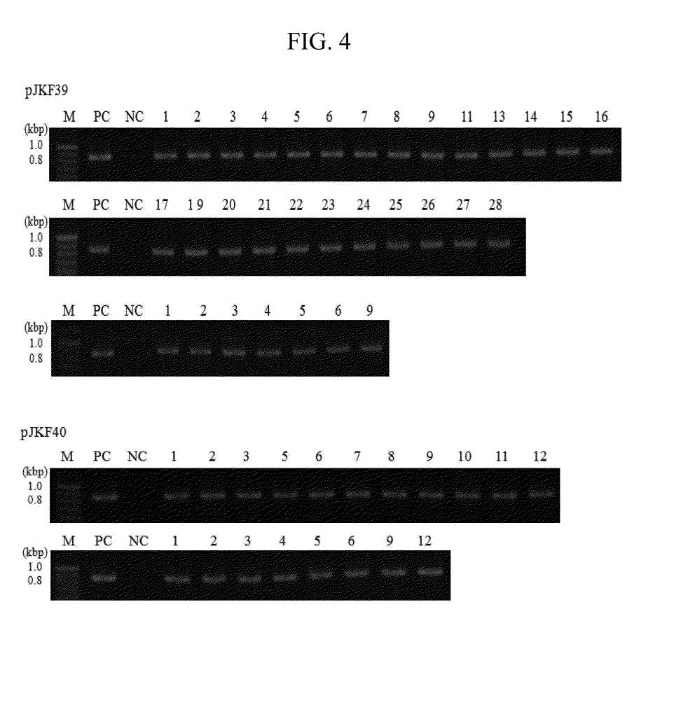

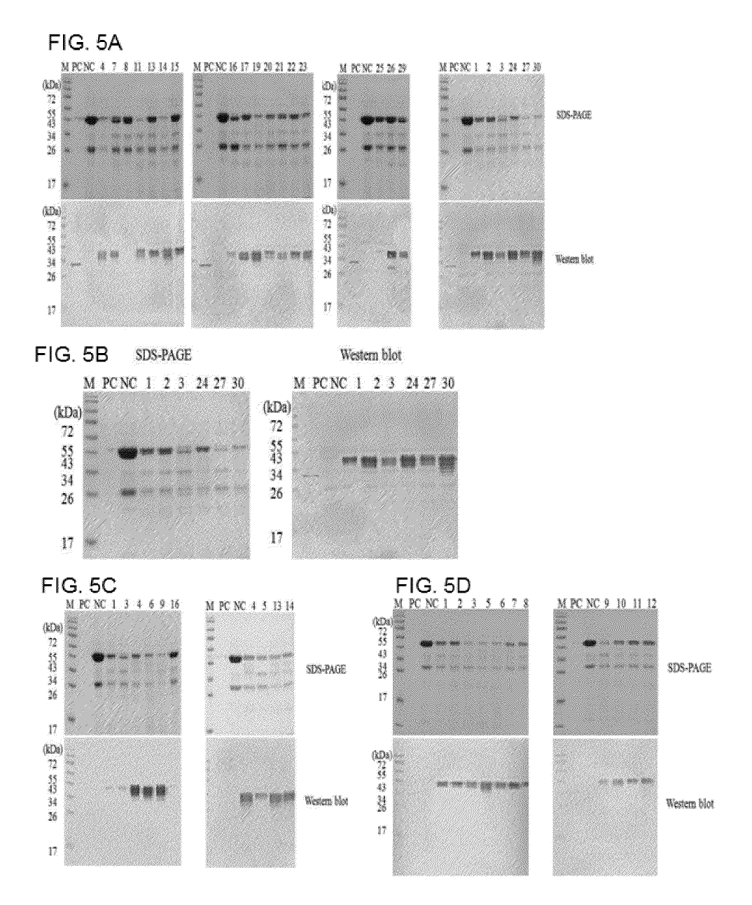

Plant transformation was determined by PCR for the genomic DNA after the gene incorporation. As a result of performing PCR, the DNA band of about 850 bp corresponding to the gene encoding the human enterokinase light chain protein as bound with the rice alpha amylase 3D signal peptide was confirmed only from the callus which was able to grow on the medium containing hygromycin (FIG. 4). Further, among 67 lines in which the callus was formed, the gene incorporation was found from 56 lines. Each callus confirmed by PCR with the gene incorporation was induced to give suspension cells for the analysis at the level of RNA and protein. Based on the Western blot analysis shown in FIGS. 5A to 5D, the cell line expressing enterokinase light chain protein at high level was selected. As a result of the Western blot analysis, it was possible to confirm that the human enterokinase light chain protein is expressed at many different levels in each of the transformed rice suspension cells while no expression of the human enterokinase light chain protein was confirmed from the wild type rice suspension cells.

### (2) RNA Extraction and Northern Blot Analysis

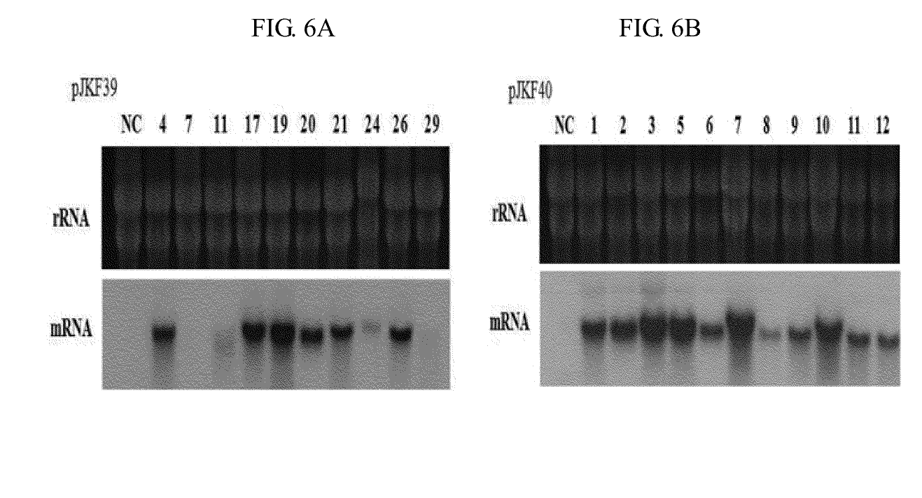

Based on the Northern blot analysis shown in FIGS. 6A and 6B, cell line expressing enterokinase light chain protein at high level was selected.

(3) Determination of Enzyme Activity of Human Enterokinase Light Chain Protein Derived from Plant

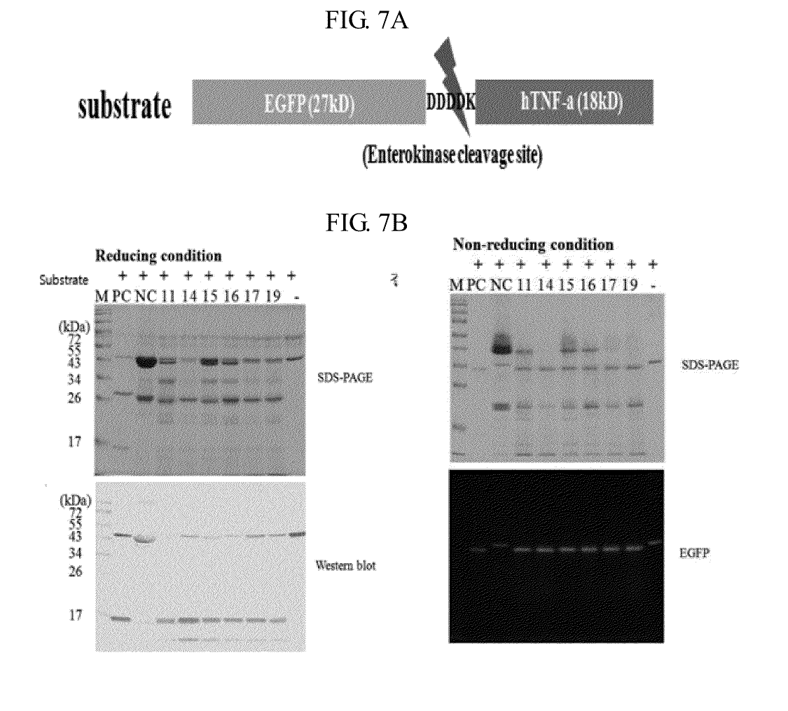

By using EGFP-hTNF-α fusion protein as a substrate, the activity of the endopeptidase activity of the enzyme from the cell line producing the cell line expressing enterokinase light chain protein at high level was determined As a result, the human enterokinase light chain protein produced from each transformed rice suspension cell exhibited the activity of the human enterokinase light chain protein, i.e., recognizing and cleaving the DDDDK amino acid sequence. However, no enzyme activity was shown from the wild type rice suspension cells (NC) (FIGS. 7A and 7B).

### EXAMPLE 5

### Establishment of Suspension Cells of Cell Line Expressing Enterokinase Light Chain Protein at High Level

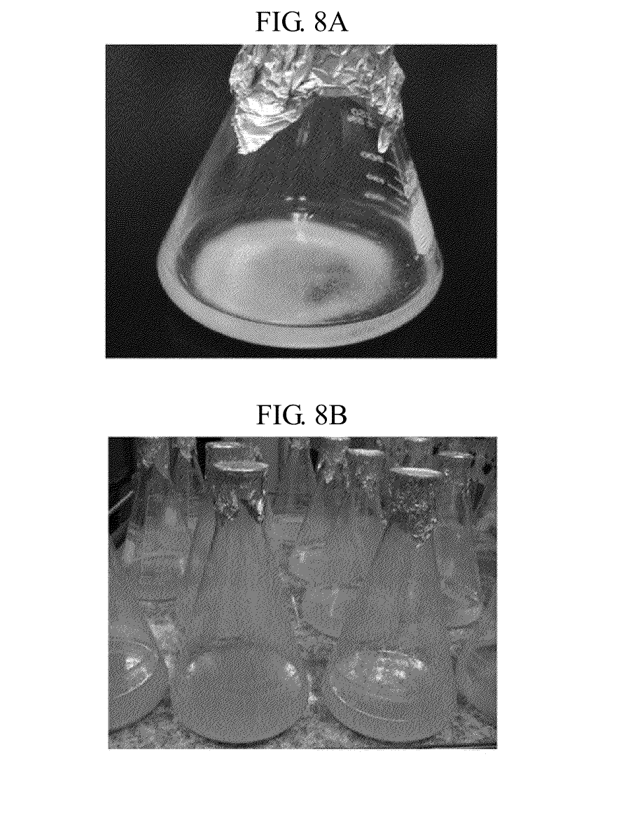

For establishing the suspension cells of cell line expressing enterokinase light chain protein at high level, suspension culture was performed by using the callus from which the expression of the gene encoding the enterokinase light chain protein has been confirmed. The pale yellow-colored callus growing in separate small areas seemed to grow relatively well on N6 selection medium although there was also a white callus appeared to be hard. After sieving with a mesh, the fine cells were subjected to sub-culture at an interval of 1 week with volume of 50 ml (FIGS. 8A and 8B). According to the passage of culture period, the cell proliferation progressed, and thus it was possible to have proliferation in volume of 300 ml in 1 liter flask (FIG. 8B).

### EXAMPLE 6

### Regeneration of Plant from Cell Line Expressing Enterokinase Light Chain Protein at High Level

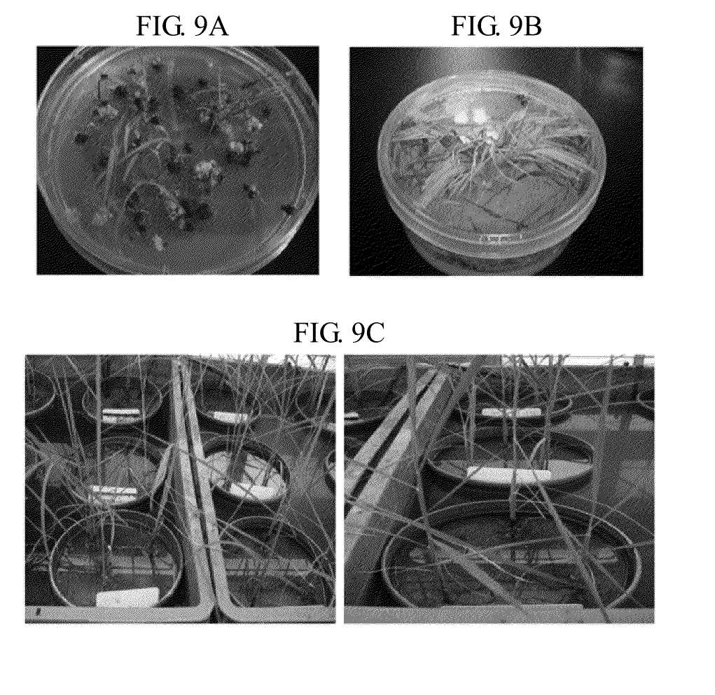

The callus from the cell line expressing enterokinase light chain protein at high level, which has been selected by Northern blot analysis and Western blot analysis, was sub-cultured in MS medium added with 0.5 mg/L kinetin and 0.05 mg/L NAA. About seven days later, it was possible to observe the formation of green premordium, and one month after having new shoots occurring two to three weeks from starting the sub-culture, root formation was observed (FIG. 9A). The plant line having regeneration on MS medium for regeneration is as follows: the line producing enterokinase light chain protein based on transformation of pJKF39 using Agrobacterium (pJKF39 a7), the line producing enterokinase light chain protein based on transformation of pJKF39 using gene gun method (pJKF39 b1, pJK39 b6, pJK39 b8, pJK39 b14 and pJKF b22), and the line producing enterokinase light chain protein based on transformation of pJKF40 using gene gun method (pJK40 b4, pJK40 b5, pJK40 b8, pJK40 b9 and pJKF b16). The young regenerated plant with new shoots and roots were sub-cultured in a culture vessel which allows height growth to induce growth into a healthy plant (FIG. 9B). The plant with fully formed roots and strong new shoots was selected, acclimated, and grown to give an adult rice plant (FIG. 9C).

### EXAMPLE 7

### Establishment of Cell Bank of Cell Line Expressing Enterokinase Light Chain Protein at High Level

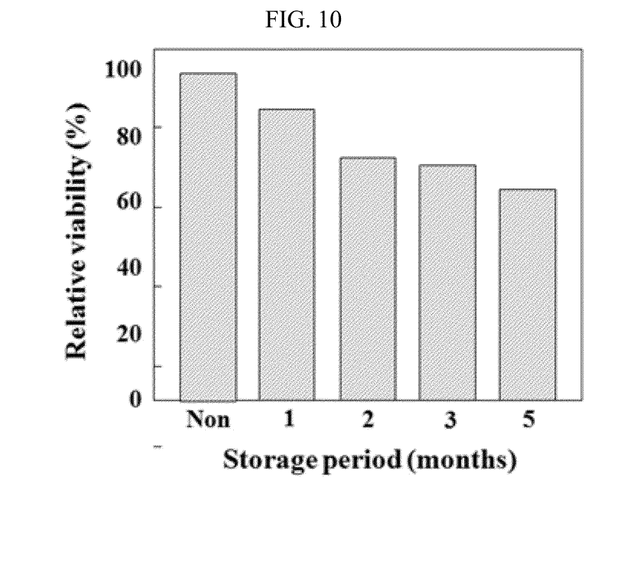

Cryopreservation of the suspension cells of rice was performed according to the method previously established by the inventors, i.e., pre-culture (5 days, 0.5M sucrose)→pre-freezing (2 hours, 1M sucrose, 1M DMSO, 1M glycerol)→re-growing (cooling rate: −0.5-0.5˜1/min)→storage for long period of time. Accordingly, the cell bank of the cell line expressing enterokinase light chain protein at high level was established. The cell line subjected to the cryopreservation was tested in terms of the cellular activity and the results are shown in FIG. 10. One month after the cryopreservation, there was the activity of 90% or higher. The activity of about 70% or higher was maintained even after five months, and thus it was able to confirm that the cell line bank was successfully established.

### EXAMPLE 8

### Large-Scale Production of Cell Line Expressing Enterokinase Light Chain Protein at High Level

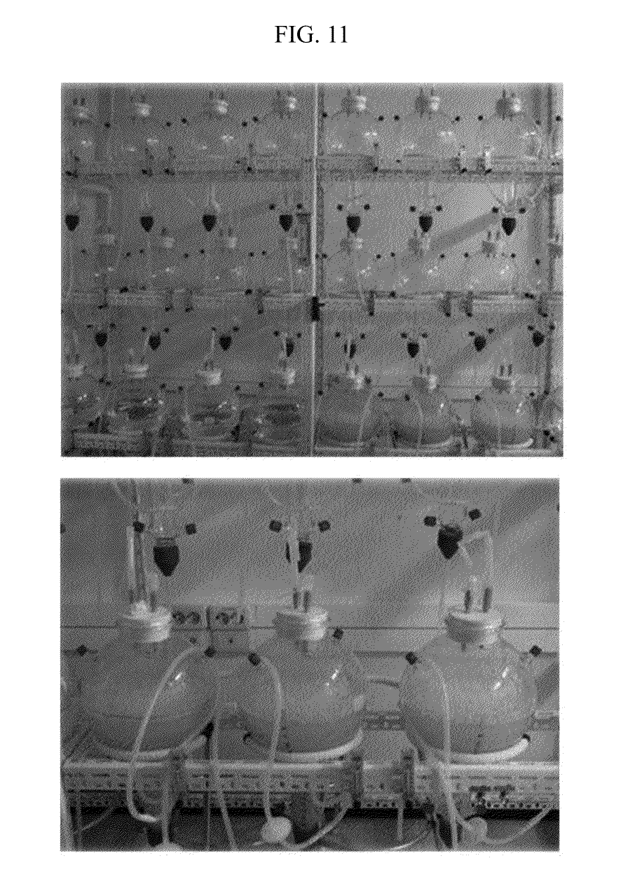

In order to establish the suspension culture system for the cell line expressing the enterokinase light chain protein at high level, expression of the gene encoding the enterokinase light chain protein was determined by Northern blot analysis and Western blot analysis, and the line showing strong callus vitality after suspension culture was selected; i.e., the enterokinase light chain protein-producing line obtained by transforming with pJKF39 using Agrobacterium (pJKF39 a3, ppJKF39 a5, pJKF39 a9), the enterokinase light chain protein-producing line obtained by transforming with pJKF39 using gene gun method (pJKF39 b11, pJKF39 b14, pJKF39 b 26, pJKF39 b30), the enterokinase light chain protein-producing line obtained by transforming with pJKF40 using Agrobacterium (pJK40 a3, pJK40 a5, pJK40 a7), and the enterokinase light chain protein-producing line obtained by transforming with pJKF40 using gene gun method (pJK40 b4, pJK40 b6). After that, in a culture room having thirty bubble type 10 liter bioreactors for large-scale culture of transformed rice suspension cells, large-scale cell culture was performed (FIG. 11). By increasing the volume of the suspension cells which have been established in volume of 50 ml, the large-scale production was performed by using a 10 liter bioreactor. Specifically, 100 ml of the cells were added to a specially manufactured 10 liter bioreactor, and 5 liter of N6 medium was added. Seven days after starting the culture, the medium was exchanged with the same but fresh medium not added with sucrose. The cells were harvested five days later.

Based on the study results shown above, pJKF39 b14 and pJKF40 b4 were selected and large-scale culture of the suspension cells was performed. According to Northern blot analysis and Western blot analysis, expression of the gene encoding the enterokinase light chain protein was confirmed and strong vitality was observed in terms of color of the callus or cell proliferation as determined after suspension culture. It was also confirmed that the cells have normal growth by having smooth proliferation. As such, they were cultured at large-scale and used as a material for isolating and purifying the human enterokinase light chain protein.

### EXAMPLE 9

### Isolation and Purification of Enterokinase Light Chain Protein

(1) Establishment of Conditions for Purifying Enterokinase Light Chain Protein

Based on the three-step purification of ammonium sulfate precipitation→hydrophobic interaction chromatography using phenyl Sepharose 6FF resin→ion exchange chromatography using mono S and mono Q, the enterokinase light chain protein produced by suspension cell culture of transformed rice can be purified with purity of 90% or higher. To 1 liter of the culture liquid containing transformed suspension cells, 472 g of ammonium sulfate (final concentration of 70%) was added, stirred for 2 to 4 hours, and centrifuged. The supernatant was collected and subjected to hydrophobic interaction chromatography using phenyl Sepharose 6FF resin (GE healthcare). While performing the hydrophobic interaction chromatography using phenyl Sepharose 6FF resin (GE healthcare), the influence of the ammonium sulfate concentration in an elution buffer which is used for extraction of the enterokinase light chain protein was determined As a result, it was found that, when the ammonium sulfate ion concentration is 0.4 M or less in an elution buffer, the yield of the entire extracted protein was significantly low. However, at the concentration of 0.4 M or higher, it was able to confirm the tendency that the yield of the entire extracted protein is high but the purity of the enterokinase light chain protein is low. Taken together the above results, for purification of the enterokinase light chain protein derived from rice cells based on hydrophobic interaction chromatography using phenyl Sepharose 6FF resin (GE healthcare), the ammonium sulfate ion concentration in an elution buffer is most suitably 0.4 M. The culture liquid first purified by ammonium sulfate precipitation was purified again by using phenyl Sepharose 6FF resin, and as a result, the enterokinase light chain protein was purified with the purity of about 80%.

(2) Purification Using Size Exclusion Chromatography and FPLC

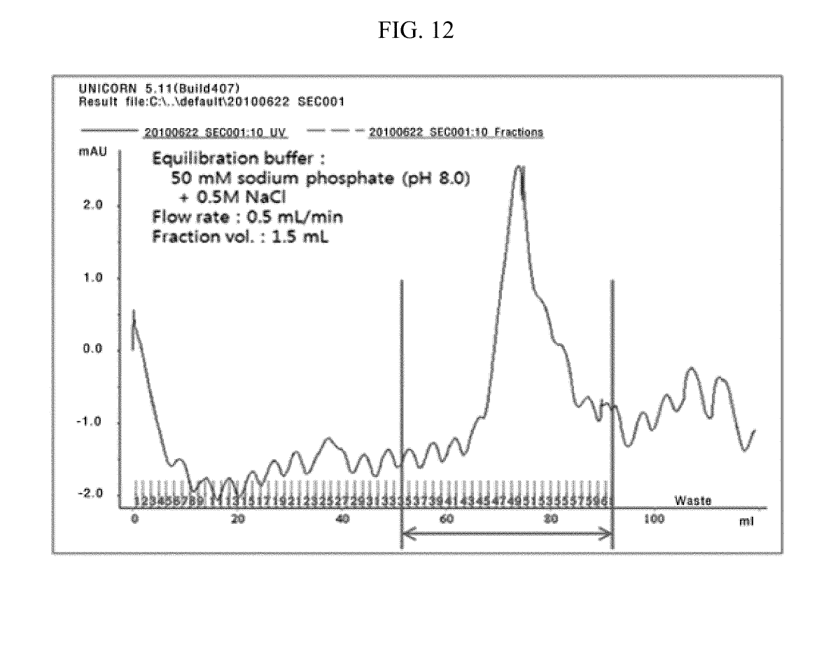

To purify the enterokinase light chain protein derived from rice suspension cells, which exhibited the purity of about 80% after purification using phenyl Sepharose 6FF resin, size exclusion chromatography using FPLC and Hiprep 16/60 Sephacryl S200 HR column (GE healthcare) was performed. The results are shown in FIG. 12. By using Hi-prep 16/60 Sephacryl S200 HR column (GE healthcare) added with FPLC, the enterokinase light chain protein showing purity of about 80% as purified first with phenyl Sepharose 6FF resin was analyzed in terms of separation degree based on UV peaks. As a result, it was confirmed that a large amount of the proteins passes through between 60 minutes and 80 minutes after injecting the sample. Thus, this range was fractionated with volume of 1 ml/min to collect eluents. By using SDS-PAGE, each eluent was tested in terms of the purity of the enterokinase light chain protein. As a result, it was possible to have efficient separation between 65 minutes and 80 minutes (i.e., Fraction number 45 to 55), and it is believed that the purity of the recombinant enterokinase light chain protein is about 90% or higher.

1. A synthetic gene encoding the human enterokinase light chain protein, which consists of the nucleotide sequence of SEQ ID NO: 1.

2. The gene according to claim 1, wherein the synthetic gene further comprises a gene encoding rice alpha amylase 3D (RAmy3D) secretion signal at the 5′-terminal.

3. The gene according to claim 1, wherein the synthetic gene further comprises a gene encoding 6× His at the 3′-terminal

4. A recombinant vector comprising the synthetic gene encoding the human enterokinase light chain protein according to any one of claims 1 to 3.

5. A plant cell transformed with the recombinant vector of claim 4.

6. A method for producing a human enterokinase light chain protein in a plant, comprising:
introducing the recombinant vector of the claim 4 to callus of a plant and selecting the callus which produces the human enterokinase light chain protein;
performing suspension culture of the selected callus; and
isolating and purifying the human enterokinase light chain protein from suspension culture liquid.

7. The method according to claim 6, wherein the plant is rice.

8. The method according to claim 6, wherein isolation and purification of the human enterokinase light chain protein is to perform ammonium sulfate precipitation, hydrophobic interaction chromatography, and ion exchange chromatography in order.

9. A method for producing a plant producing the human enterokinase light chain protein, comprising:
transforming a plant cell with the recombinant vector comprising the synthetic gene encoding the human enterokinase light chain protein according to any one of claims 1 to 3; and
regenerating a plant from the transformed plant cell.

10. A plant producing the human enterokinase light chain protein which is produced by the method of claim 9.

11. The plant according to claim 10, wherein the plant is a monocot plant.

12. The plant according to claim 11, wherein the monocot plant is rice.

13. A seed of the plant of claim 10.

14. A composition for large-scale production of the human enterokinase light chain protein in a plant, comprising the synthetic gene encoding the human enterokinase light chain protein according to any one of claims 1 to 3.
